(19) United States
(12) Patent Application Publication
(10) Pub. No.: US 2014/0271544 A1
(43) Pub. Date: Sep. 18, 2014

(54) COMBINATION THERAPY FOR TREATING HCV INFECTION IN SPECIFIC PATIENT SUBGENOTYPE SUB-POPULATION

(71) Applicants: Wulf Otto BOECHER, Biberach an der Riss (DE); Gerhard NEHMIZ, Biberach an der Riss (DE); Wiebke SAUTER, Memmingen (DE); Gerhard G. STEINMANN, Erbach (DE); Heike ZIMDAHL-GELLING, Biberach an der Riss (DE)

(72) Inventors: Wulf Otto BOECHER, Biberach an der Riss (DE); Gerhard NEHMIZ, Biberach an der Riss (DE); Wiebke SAUTER, Memmingen (DE); Gerhard G. STEINMANN, Erbach (DE); Heike ZIMDAHL-GELLING, Biberach an der Riss (DE)

(73) Assignee: Boehringer Ingelheim International GmbH, Ingelheim an Rhein (DE)

(21) Appl. No.: 13/799,133

(22) Filed: Mar. 13, 2013

(51) Int. Cl. A61K 31/4709; A61K 31/7056; A61K 38/21

(52) U.S. Cl. CPC A61K 31/4709; A61K 38/212; A61K 31/7056
USPC 424/85.7

(57) ABSTRACT

The present invention relates to therapeutic combinations comprising (a) Compound (1), or a pharmaceutically acceptable salt thereof, as herein described, (b) an interferon alfa and (c) ribavirin. Compound (1) is a selective and potent inhibitor of the HCV NS3 serine protease. The present invention also relates to methods of using such therapeutic combinations for treating HCV infection or alleviating one or more symptoms thereof in patients having genetic variations located near the IL28B gene, including SNP rs12979860 with a non-CC genotype and SNP rs8099917 with a non-TT genotype.

### TECHNICAL FIELD OF THE INVENTION

The present invention relates to therapeutic combinations comprising Compound (1) as herein described, an interferon alfa and ribavirin. The present invention also relates to methods of using such therapeutic combinations for treating HCV infection or alleviating one or more symptoms thereof in a patient that has been identified as having genetic variations located near the IL28B gene, including SNP rs12979860 with a non-CC genotype and SNP rs8099917 with a non-TT genotype. The present invention also provides kits comprising the therapeutic combinations of the present invention.

### BACKGROUND OF THE INVENTION

The following Compound (1):

[chemical structure]

wherein B is

[chemical structure]

### L⁰ is MeO—; L¹ is Br; and R² is

[chemical structure]

having the chemical name: 1-{[4-[8-Bromo-2-(2-isopropylcarbamoyl-thiazol-4-yl)-7-methoxy-quinolin-4-yloxy]-1-(R)-(2-cyclopentyloxycarbonyl amino-3,3-(S)-dimethyl-butyryl)-pyrrolidine-(S)-2-carbonyl]-amino}-2-(S)-vinyl-cyclopropane-(R)-carboxylic acid, also known as faldaprevir, is known as a selective and potent inhibitor of the HCV NS3 serine protease and useful in the treatment of HCV infection. Compound (1) falls within the scope of the acyclic peptide series of HCV inhibitors disclosed in U.S. Pat. Nos. RE 40,525, 7,514,557 and 7,585,845. Compound (1) is disclosed specifically as Compound #1055 in U.S. Pat. No. 7,585,845, and as Compound #1008 in U.S. Pat. No. 7,514,557. Compound (1), and pharmaceutical formulations thereof, can be prepared according to the general procedures found in the above-cited references, all of which are herein incorporated by reference in their entirety. Preferred forms of Compound (1) include the crystalline forms, in particular the crystalline sodium salt form, which can be prepared as described in U.S. Pat. No. 8,232,293, also incorporated herein by reference.

Data demonstrating the activity of Compound (1) as an inhibitor of the HCV NS3 serine protease and its corresponding demonstrated utility in the treatment of HCV infection in patients, can be found in U.S. Pat. No. 7,585,845, as well as in numerous publications presenting the preclinical characterization or clinical trial results with Compound (1). See, e.g., Sulkowski M S, et al, Hepatol (2009), Vol. 50, pg. 2A, Abstract LB3; Sulkowski M S, et al., J Hepatol (2010) Vol. 52, Supp. 1, pgs. S462-S463, Abstract 1190; Berg et al., Hepatol (2010), Vol. S2, Supp. S1, Abstract 804; and White P W, et al., Antimicrob Agents Chemother (2010) 54(11):4611-4618.

Combination therapy regimens directed to administering Compound (1) with an interferon-alpha and ribavirin for the treatment of HCV infection are described in U.S. Patent Application Publication Nos. 2010/0068182 and 2011/0268700.

It is known in the art that particular HCV subtypes and patient subgenotypes may respond differently to HCV therapy. HCV Genotype 1a is traditionally more difficult to treat and are less responsive to antiviral therapy than Genotype 1b. See, e.g., Ghany, Marc et al. “An Update on Treatment of Genotype 1 Chronic Hepatitis C Virus Infection: 2011 Practice Guideline by the American Association for the Study of Liver Diseases”, Hepatology, 54(4): 1433-44 (2011)). In addition, and particularly with interferon-based therapy, specific single nucleotide polymorphisms (SNPs) located on the long arm of chromosome 19 within the gene cluster of IL-28B (Interleukin (IL) 28B, also called lambda interferon), of the patient undergoing therapy can directly effect the responsiveness of that patient to the antiviral therapy. In particular, patients having a non-CC genotype of SNP rs12979860 or a non-TT genotype of SNP rs8099917 are traditionally more difficult to treat and are less responsive in terms of a sustained virological response (SVR) than patients having the CC or TT genotype. The SNP that was most strongly associated with SVR in the genome-wide analysis was rs12979860 followed by rs8099917. See, e.g., Ge et al., Nature, 461:399-401 (2009) and Balagopal, Gastroenterology, 139:1865-1876 (2010), Some studies showed an almost seven fold difference in treatment response. See G. Cairns, “Gene variant that helps hepatitis C treatment may hinder HIV treatment”, 2011, at: http://www.bhiva.org/News.aspx?NewsID=a7503829-94b9-4-d2f-bd91-1d2fbaad6e8d.

It is further known that this difference in SVR rates and cure of disease did not change when a NS3/4 HCV protease inhibitor (telaprevir) was added to the standard regimen with pegylated interferon alfa and ribavirin (PegIFN/RBV). As shown by Akuta et al., Hepatology, 52: 421-429 (2010), patients with a non-CC or non-TT genotype experienced SVR rates that differed strongly by 51.5% or 56.2% compared to CC or TT genotypes according to the genetic variation in rs12979860 or rs8099917, respectively, located near the IL28B gene. Finally, IL28B genotype associations have also been found with early viral kinetics during interferon free treatment of HCV patients. See Chu et al., “Effect of IL28B Genotype on Early Viral Kinetics During Interferon-Free Treatment of Patients With Chronic Hepatitis C”, Gastroenterology (2012), currently in press, available online Jan. 13, 2012.

Thus, there is a need in the art for therapies that are effective against even the more difficult-to-treat patient subpopulations, particularly those exhibiting HCV subtype 1a and having a non-CC genotype of SNP rs12979860 or a non-TT genotype of SNP rs8099917 located near the IL28B gene.

### BRIEF SUMMARY OF THE INVENTION

It has now been discovered that the combination of Compounds (1), or a pharmaceutically acceptable salt thereof, with interferon alpha and ribavirin, have good effectiveness in treating even the traditionally difficult-to-treat HCV patient subpopulations, particularly those patients with a non-CC genotype of SNP rs12979860 or a non-TT genotype of SNP rs8099917, both located near the IL28B gene.

It is surprising that the expected IL28B-related difference in SVR rates that have been observed in patients treated with PegIFN/RBV and the NS3/4 HCV protease inhibitor telaprevir widely disappeared when using the therapeutic combination of the present invention and that patients with the unfavorable genotypes non-CC or non-TT or with the unfavorable HCV subtype 1a infections achieved consistently high cure rates in terms of SVR.

The present invention provides a method of treating HCV infection or alleviating one or more symptoms thereof in a patient comprising the step of administering to the patient a therapeutic combination comprising a Compound (1) as defined herein, or a pharmaceutically acceptable salt thereof, together with an interferon alfa and ribavirin, as defined herein, and wherein the patient has a non-CC genotype of SNP rs12979860 or a non-TT genotype of SNP rs8099917 located near the IL28B gene. The three actives of the combination can be administered simultaneously or separately, as part of a regimen.

The present invention further provides for a packaged pharmaceutical composition comprising a packaging containing one or more doses of Compound (1), or a pharmaceutically acceptable salt thereof and (b) written instructions directing the co-administration of Compound (1), or a pharmaceutically acceptable salt thereof, interferon alpha, and ribavirin for the treatment of HCV infection in a patient that has a non-CC genotype of SNP rs12979860 or a non-TT genotype of SNP rs8099917 located near the IL28B gene.

### DETAILED DESCRIPTION OF THE INVENTION

### Definitions

“Compound (1)” is as defined above.

“HCV infection” as used herein means infection by any subtype of the Hepatitis C Virus, including subtypes 1-6, and includes both acute and chronic HCV infection.

“Interferon” means a member of a family of highly homologous species-specific proteins that inhibit viral replication and cellular proliferation and modulate immune response. Human interferons are grouped into three classes based on their cellular origin and antigenicity: α-interferon (leukocytes), β-interferon (fibroblasts) and γ-interferon (B cells). Recombinant forms of each group have been developed and are commercially available. Subtypes in each group are based on antigenic/structural characteristics. At least 24 interferon alfas (grouped into subtypes A through H) having distinct amino acid sequences have been identified by isolating and sequencing DNA encoding these peptides. The terms “α-interferon”, “alfa-interferon” and “interferon alfa” are used interchangeably in this application to describe members of this group. Both naturally occurring and recombinant alfa-interferons, including consensus interferon, may be used in the practice of the invention.

Suitable interferon-alfas for the present invention include, but are not limited to, recombinant interferon alfa-2b such as INTRON®-A interferon and VIRAFERON®; recombinant interferon alfa-2a such as ROFERON® interferon; recombinant interferon alfa-2c such as BEROFOR® alfa 2 interferon; interferon alfa-n1, a purified blend of natural alfa interferons such as SUMIFERON® or WELLFERON® interferon alfa-n1 (INS); or a consensus alfa interferon such as those described in U.S. Pat. Nos. 4,897,471 and 4,695,623; or interferon alfa-n3, a mixture of natural alfa interferons such as ALFERON®. The use of interferon alfa-2a or alfa 2b is preferred. The manufacture of interferon alfa 2b is described in U.S. Pat. No. 4,530,901.

The term “interferon alfa” is further intended to include those “pegylated” analogs meaning polyethylene glycol modified conjugates of interferon alfa, preferably interferon alfa-2a and -2b. The preferred polyethylene-glycol-interferon alfa-2b conjugate is PEG₁₂₀₀₀-interferon alfa 2b. The term “PEG₁₂₀₀₀-IFN alfa” as used herein means conjugates such as are prepared according to the methods of International Application No. WO 95/13090 and containing urethane linkages between the interferon alfa-2a or -2b amino groups and polyethylene glycol having an average molecular weight of 12000.

The preferred PEG₁₂₀₀₀-interferon alfa-2b is prepared by attaching a PEG polymer to the epsilon amino group of a lysine residue in the IFN alfa-2b molecule. A single PEG₁₂₀₀₀ molecule is conjugated to free amino groups on an IFN alfa-2b molecule via a urethane linkage. This conjugate is characterized by the molecular weight of PEG₁₂₀₀₀ attached. The PEG₁₂₀₀₀-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

Especially preferred conjugates of interferon alfa that may be used in the present invention are pegylated alfa-interferons, e.g., pegylated interferon alfa-2a, pegylated interferon alfa-2b, pegylated consensus interferon or pegylated purified interferon alfa product. Pegylated interferon alfa-2a is described, e.g., in European Patent No. EP 0 593 868 and commercially-available, e.g., under the tradename PEGASYS® (Hoffmann-La Roche). Pegylated interferon alfa-2b is described, e.g., in U.S. Pat. No. 5,908,621 and WO 98/48840 and commercially-available, e.g., under the tradename PEG-INTRON® A (Schering Plough). Pegylated consensus interferon is described in WO 96/11953. The preferred pegylated alfa interferons are pegylated interferon alfa-2a and pegylated interferon alfa-2b. Also preferred is pegylated consensus interferon.

The term “interferon alfa” further includes other interferon alfa conjugates that can be prepared by coupling an interferon alfa to a water-soluble polymer. A non-limiting list of such polymers includes other polyalkylene oxide homopolymers such as polyethylene glycol (PEG), polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090.

The term “interferon alfa” further includes fusion proteins of an interferon alfa, for example fusion proteins of interferon-α-2a, interferon-α-2b, consensus interferon or purified interferon-α product, each of which is fused with another protein. Certain preferred fusion proteins comprise an interferon (e.g., interferon-α-2b) and an albumin as described in U.S. Pat. No. 6,972,322 and international publications WO2005/003296 and WO2005/077042. Also included are consensus interferons, such as INFERGEN®.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

“Ribavirin” refers to 1-β-D-ribofuranosyl-1H-1,2,4-triazole-3-carboxamide, available from ICN Pharmaceuticals, Inc., Costa Mesa, Calif. and is described in the Merck Index, to compound No. 8199, Eleventh Edition. Its manufacture and formulation is described in U.S. Pat. No. 4,211,771. Preferred marketed ribavirin products include REBETOL® and COPEGUS®. The term further includes derivatives or analogs thereof, such as those described in U.S. Pat. Nos. 6,063,772, 6,403,564 and 6,277,830. For example, derivatives or analogs include modified ribavirins such as 5′-amino esters, ICN Pharmaceutical's L-enantiomer of ribavirin (ICN 17261), 2′-deoxy derivatives of ribavirin and 3-carboxamidine derivatives of ribavirin, viramidine (previously known as ribamidine) and the like.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

### EMBODIMENTS OF THE INVENTION

According to a general embodiment, the present invention provides for a method of treating HCV infection or alleviating one or more symptoms thereof in a patient comprising the step of administering to the patient a therapeutic combination comprising a Compound (1) as defined herein, or a pharmaceutically acceptable salt thereof, together with an interferon alfa and ribavirin and wherein the patient has a non-CC genotype of SNP rs12979860 or a non-TT genotype of SNP rs8099917 located near the IL28B gene. In another embodiment, the present invention teaches the use of a Compound (1) as defined herein, or a pharmaceutically acceptable salt thereof, an interferon alfa, and ribavirin for the preparation of a pharmaceutical kit to treat a hepatitis C viral (HCV) infection or alleviating one or more symptoms thereof in a patient and wherein the patient has a non-CC genotype of SNP rs12979860 or a non-TT genotype of SNP rs8099917 located near the IL28B gene.

In administering the therapeutic combinations of the present invention, each active agent can be administered together at the same time or separately at different times in separate dosage administrations. The present invention contemplates and includes all such dosage regimens when administering the triple therapeutic combinations as defined herein.

Although this combination therapy is expected to be effective against all HCV genotypes, it has been demonstrated to be particularly effective in treating HCV genotype 1 infection, including subtypes 1a and 1b, and also having a non-CC genotype of SNP rs12979860 or a non-TT genotype of SNP rs 8099917 located near the IL28B gene. Particular embodiments include the following patient sub-populations:
(1) HCV subtype 1a and C/T genotype of SNP rs12979860
(2) HCV subtype 1a and T/T genotype of SNP rs12979860
(3) HCV subtype 1b and C/T genotype of SNP rs12979860
(4) HCV subtype 1b and T/T genotype of SNP rs12979860
(5) HCV subtype 1a and G/T genotype of SNP rs8099917
(6) HCV subtype 1a and G/G genotype of SNP rs8099917
(7) HCV subtype 1b and G/T genotype of SNP rs8099917
(8) HCV subtype 1b and G/G genotype of SNP rs8099917

A preferred embodiment is directed to the treatment of patients have the HCV subtype 1a and either the C/T or T/T genotype of SNP 12979860 or the G/T or G/G genotype of SNP rs8099917 located near the IL28B gene, which represent particularly difficult-to-treat HCV-infected patient populations.

In a specific preferred sub-embodiment, the patient has first been identified as having a non-CC genotype of SNP rs12979860 or a non-TT genotype of SNP rs8099917 located near the IL-28B gene prior to the step of administering the therapeutic combination of the present invention. Methods for such genotypic identification as are set forth in detail herein.

The patient population to be treated with the combination therapy of the present invention can be further classified into “treatment-naïve” patients, i.e., those patient who have not received any prior treatment for HCV infection and “treatment experienced” patients, i.e, those patients who have undergone prior treatment for HCV. Either of these classes of patients may be treated with the combination therapy of the present invention. The clinical data presented hereinafter is directed to treatment naïve patients only. Nevertheless, there is an expectation that similar efficacy results will be seen in treatment experienced patients. A particular class of patients that are preferably treated are those treatment experienced patients that have undergone prior interferon plus ribavirin therapy but are non-responsive to said therapy (herein “non-responders”). Such non-responders include three distinct groups of patients: (1) those who experienced<1×log₁₀ maximum reduction in HCV RNA levels during treatment with interferon plus ribavirin (“null responders”), (2) those who experienced≧1×logᵢₙ maximum reduction in HCV RNA levels during treatment with interferon plus ribavirin but never achieve HCV RNA levels below level of detection (“partial responders”), and (3) those who achieved undetectable HCV RNA levels with and during interferon plus ribavirin therapy but had a viral load rebound after treatment has completed (“relapser”). Another treatment experienced patient population to be treated with the combination therapy of the present invention includes those who achieved an initial virologic response with (pegylated) interferon plus ribavirin but had viral load rebound during treatment other than due to nonadherence to the treatment.

According to an alternative embodiment, the present invention provides a method of reducing HCV-RNA levels in a patient in need thereof, comprising the step of administering to said patient a therapeutic combination according to the present invention.

Preferably, the method of the present invention reduces the HCV-RNA levels in a patient to a level below the lower limit of quantification (or “BLQ”). A BLQ level of HCV RNA as used in the present invention means a level below 25 International Units (IU) per ml of serum or plasma of a patient as measured by quantitative, multi-cycle reverse transcriptase PCR methodology according to the WHO international standard (Saladanha J, Lelie N and Heath A, Establishment of the first international standard for nucleic acid amplification technology (NAT) assays for HCV RNA. WHO Collaborative Study Group. Vox Sang 76:149-158, 1999). Such methods are well known in the art. In a preferred embodiment, the method of the present invention reduces the HCV-RNA levels in a patient to less than 25 IU per ml of serum or plasma. In another embodiment the method of the present invention reduces the HCV-RNA levels in a patient to less than a detectible level.

In a preferred embodiment, the method of the present invention reduces the HCV-RNA levels in a patient to less than 25 IU per ml of serum, even more preferably to less than 10 IU per ml of serum.

In another embodiment the method of the present invention reduces the HCV-RNA levels in a patient to less than a detectible level (below the limit of detection, BLD). Treatment decisions for duration of HCV therapy can be made based on BLD, and combinations of BLQ and BLD HCV RNA at subsequent timepoints during initial treatment. Typical time points include HCV RNA measurements at 4, 8, and 12 weeks after initiation of therapy, and results are utilized to guide further treatment duration “response-guided therapy”. Cure from HCV infection is typically inferred if HCV RNA remained BLD 12-24 weeks after end of HCV treatment. Thus, in additional embodiments, the method of the present invention results in an HCV-RNA level in the patient that is less than a detectible level at 12 weeks, preferably 24 weeks, after the end of all treatment.

The usual duration of the treatment for standard interferon plus ribavirin therapy is at least 48 weeks, and up to 72 weeks, for chronic infection with HCV genotype 1 or 4; 48 weeks for the majority of patients with chronic HCV genotype 2 or 3 infection. A few patients with chronic HCV genotype 2 and 3 infection may be treated with 24 weeks of interferon alpha and ribavirin. However, with the addition of Compound (1), or a pharmaceutically acceptable salt thereof, in the triple combination therapy of the present invention, it may be possible to have a much shorter duration of treatment. With the triple combination therapy of the present invention the contemplated durations of treatment include at least 4 weeks, preferably at least 12 weeks, e.g., from about 12 weeks to about 24 weeks, although treatment up to and even beyond 48 weeks is possible as well. Thus, further embodiments include treatment for at least 24 weeks and for at least 48 weeks. The duration of treatment of chronic HCV infection may vary depending upon the specific HCV genotype. For example, the typical duration of treatment will be longer for genotypes 1 and 4, than for genotypes 2 and 3. In addition, the treatment duration will be shorter for the treatment of acute infection as compared to chronic infection. Also contemplated is an initial treatment regimen with the triple combination therapy of the present invention, followed by a continuation of only the interferon plus ribavirin double combination therapy. Thus, possible scenarios for the initial triple and then double combination therapy include, for example: (1) 4 weeks of the triple combination therapy, followed by 8 to 44 weeks of the interferon plus ribavirin only therapy; (2) 12 weeks of the triple combination therapy, followed by 0 to 36 weeks of the interferon plus ribavirin only therapy; and (3) 24 weeks of the triple combination therapy, followed by 0 to 24 weeks of the interferon plus ribavirin only therapy.

The first component of the therapeutic combination, namely, Compound (1) or a pharmaceutically acceptable salt thereof is comprised in a composition. Such a composition comprises Compound (1), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,585,845, WO 2010/059667 and WO 2011/005646.

In general, the Compound (1) or a pharmaceutically acceptable salt thereof may be administered at a maintenance dosage of at least 40 mg/day (in single or divided doses). Additional embodiments for dosage amounts and ranges may include (in single or divided doses):
(a) at least 48 mg/day
(b) at least 100 mg/day
(c) at least 120 mg/day
(d) at least 200 mg/day
(e) at least 240 mg/day
(f) at least 360 mg/day
(g) at least 480 mg/day
(h) from about 40 mg/day to about 480 mg/day
(i) from about 48 mg/day to about 240 mg/day
(j) from about 100 mg/day to about 300 mg/day
(k) from about 120 mg/day to about 300 mg/day
(l) from about 120 mg/day to about 240 mg/day
(m) from about 240 mg/day to about 480 mg/day
(n) about 48 mg/day
(O) about 120 mg/day
(p) about 240 mg/day
(q) about 360 mg/day
(r) about 480 mg/day

Although Compound (1) or a pharmaceutically acceptable salt thereof may be administered in single or divided daily doses, once a day administration (QD) of the daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combinations (co-medications), the severity and course of the infection, the patient's disposition to the infection and the judgment of the treating physician. Specific factors affecting dosing may include, for example, individual patient factors which modify the adsorption, distribution, metabolism and excretion of Compound (1); the specific HCV Genotype; the specific IL28B genotype of the patient; the patient's innate/adaptive immune response to HCV; acute vs. chronic HCV infection; and the disposition of ribavirin based on host factors. In general, the compound is most desirably administered at a concentration level that will generally afford antivirally effective results without causing any harmful or deleterious side effects.

In another embodiment according to the invention, a loading dose amount of Compound (1) is administered for the first administration dose of the treatment. The loading dose amount is higher than the dose amount administered for subsequent administrations in the treatment, which are referred to as maintenance doses. Preferably, the loading dose amount is about double in quantity, by weight, of the amount in subsequent administrations in the treatment. For example, in one embodiment, the first dose of Compound (1) administered at a loading dosage of about 240 mg and subsequent maintenance doses of Compound (1) are administered at a dosage of about 120 mg. In another embodiment, the first dose of Compound (1) administered at a loading dosage of about 480 mg and subsequent maintenance doses of Compound (1) are administered at a dosage of about 240 mg. By using this loading dose concept, a clear advantage is that it is thereby possible to achieve steady state levels of active drug in the patient's system earlier than would otherwise be achieved. A higher blood level is achieved early by using a loading dose preferably double the maintenance dose at first intake. Reaching the targeted steady state level of active drug earlier in therapy also means that there is less possibility of insufficient drug exposure at the beginning of therapy so that resistant viral strains have a smaller chance of emerging.

The second component of the therapeutic combination, namely interferon-alfa, is comprised in a pharmaceutical composition. Typically, such compositions are injectible formulations comprising interferon-alfa and a pharmaceutically acceptable adjuvant or carrier and are well known in the art, including in a number of marketed interferon-alfa formulations. See, e.g., the various marketed interferon-alfa products and various patent and other literature related to interferon-alfa cited hereinabove.

The types of interferon-alfas that may be used in the combination are as outlined hereinabove in the definitions section. In one preferred embodiment, the interferon alfa is a pegylated interferon alfa. In a further embodiment, the interferon alfa is a pegylated interferon alfa-2a or pegylated interferon alfa-2b. In a particularly preferred embodiment, the interferon alfa is PEGASYS® or PEG-INTRON®.

When using known, marketed interferon alfa products, such products may be administered at their labeled dosage levels indicated for interferon plus ribavirin combination therapy for the treatment of HCV infection. Of course, with the triple combination therapy of the present invention it may be possible to use a lower dosage of interferon alfa, e.g., significantly lower than is used the current standard interferon plus ribavirin therapy, while delivering the same or better efficacy than the current standard therapy with less side-effects usually associated with such therapy.

In one embodiment, the interferon alfa may be administered parenterally one to three times per week, preferably once or twice a week. With respect to pegylated interferon alfas, these are typically administered once per week and the total weekly dose ranges, e.g., from about 0.5 μg/kg/week to about 2 μg/kg/week in case of pegylated interferon alfa-2b, and with respect to pegylated interferon alfa-2a the dosage is independent from the body weight of the host and is typically about 90 to 200 μg/week, more preferably about 160 to about 200 μg/week. In combination with ribavirin, a standard dosage of pegylated interferon alfa-2b is about 1.5 μg/kg/week and a standard dosage of pegylated interferon alfa-2a is about 180 μg/week, together with ribavirin, which is preferably dosed once or twice daily according to body weight and with a total daily dose of about 200-1800 mg/day, in particular, 800-1200 mg/day of oral ribavirin.

According to further embodiments, the pegylated interferon alfa-2b may be administered at dosages of:
(a) about 0.5 μg/kg/week to about 2 μg/kg/week;
(b) about 1 μg/kg/week to about 2 μg/kg/week;
(c) about 1.5 μg/kg/week to about 2 μg/kg/week;
(d) about 1.5 μg/kg/week

According to further embodiments, the pegylated interferon alfa-2a may be administered at dosages of:
(a) about 90 to about 200 μg/week;
(b) about 160 to about 200 μg/week;
(b) about 180 μg/week

The third component of the therapeutic combination, namely ribavirin, is comprised in a pharmaceutical composition. Typically, such compositions comprise ribavirin and a pharmaceutically acceptable adjuvant or carrier and are well known in the art, including in to a number of marketed ribavirin formulations. Formulations comprising ribavirin are also disclosed, e.g., in U.S. Pat. No. 4,211,771.

The types of ribavirin that may be used in the combination are as outlined hereinabove in the definitions section. In one preferred embodiment, the ribavirin is either REBETOL® or COPEGUS® and they may be administered at their labeled dosage levels indicated for interferon plus ribavirin combination therapy for the treatment of HCV infection. Of course, with the triple combination therapy of the present invention it may be possible to use a lower dosage of ribavirin, e.g., lower than is used the current standard interferon plus ribavirin therapy, while delivering the same or better efficacy than the current standard therapy with less side-effects usually associated with such therapy.

According to various embodiments, the ribavirin may be administered at dosages of (in single or divided doses):
(a) between 200 mg/day to about 1800 mg/day;
(b) between about 800 mg/day to about 1200 mg/day;
(c) between about 1000 mg/day to about 1200 mg/day;
(d) about 1000 mg/day
(e) about 1200 mg/day
(f) between about 300 mg/day to about 800 mg/day
(g) between about 300 mg/day to about 700 mg/day
(h) between 500 mg/day to about 700 mg/day
(i) between 400 mg/day to about 600 mg/day
(j) about 400 mg/day
(k) about 600 mg/day
(l) about 800 mg/day

According to one embodiment, the ribavirin composition comprises ribavirin in a formulation suitable for dosing once a day or twice daily. For example, if a therapeutic combination comprises about 1000 mg/day dosage of ribavirin, and a dosing of two times a day is desired, then the therapeutic combination will comprise ribavirin in a formulation, e.g., a tablet, containing, e.g., about 200 mg of ribavirin, with the first dose of 600 mg (or 400 mg), followed by a second dose of 400 mg (or 600 mg) at least 6 hours apart.

With respect to the Compound (1) or a pharmaceutically acceptable salt thereof plus interferon alfa plus ribavirin triple combination therapy of the present invention, the present invention contemplates and includes all combinations of the various preferred embodiments and sub-embodiments as set forth hereinabove.

For example, in one embodiment the present invention contemplates a method of treating hepatitis C viral (HCV) infection or alleviating one or more symptoms thereof in a patient that has the non-CC genotype of SNP rs12979860 or a non-TT genotype of SNP rs8099917 located near the IL28B gene comprising the step of administering to the patient a therapeutic combination comprising:
(a) Compound (1) or a pharmaceutically acceptable salt thereof at a dosage between about 40 mg per day and about 480 mg per day;
(b) pegylated interferon alfa-2a at a dosage of about 160 to about 200 μg/week or pegylated interferon alfa-2b at a dosage of about 0.5 μg/kg/week to about 2 μg/kg/week; and
(c) ribavirin at a dosage of between about 200 mg/day to about 1800 mg/day.

In another embodiment the present invention contemplates a method of treating hepatitis C viral (HCV) infection or alleviating one or more symptoms thereof in a patient that has the non-CC genotype of SNP rs12979860 or a non-TT genotype of SNP rs8099917 located near the IL28B gene comprising the step of administering to the patient a therapeutic combination comprising:
(a) Compound (1) or a pharmaceutically acceptable salt thereof at a dosage between about 120 mg per day and about 240 mg per day;
(b) pegylated interferon alfa-2a at a dosage of about 180 μg/week; and
(c) ribavirin at a dosage of between about 1000 mg/day to about 1200 mg/day.

In another embodiment the present invention contemplates a method of treating hepatitis C viral (HCV) infection or alleviating one or more symptoms thereof in a patient that has the non-CC genotype of SNP rs12979860 or a non-TT genotype of SNP rs8099917 located near the IL28B gene comprising the step of administering to the patient a therapeutic combination comprising:
(a) Compound (1) or a pharmaceutically acceptable salt thereof at a dosage between about 120 mg per day and about 240 mg per day;
(b) pegylated interferon alfa-2b at a dosage of about 1.5 μg/kg/week; and
(c) ribavirin at a dosage of between about 1000 mg/day to about 1200 mg/day.

Further embodiments include any of the above-mentioned embodiments, and where:
(a) the HCV infection is genotype 1, preferably genotype 1a, and the patient is a treatment-naïve patient; or
(b) the HCV infection is genotype 1, preferably genotype 1a, and the patient is a treatment-experienced patient who is non-responsive to a combination therapy of interferon plus ribavirin.

In further embodiments, the patient has first been identified as having a non-CC genotype of SNP rs12979860 or a non-TT genotype of SNP rs8099917 located near the IL-28B gene prior to the step of administering the therapeutic combination of the present invention.

Further embodiments include any of the above-mentioned embodiments, and where the Compound (1) or a pharmaceutically acceptable salt thereof is administered once a day, the interferon alpha is administered once a week and the ribavirin is administered twice a day.

With respect to the triple combination therapies of the present invention, the present to invention contemplates and includes all combinations of the various preferred embodiments and sub-embodiments as set forth herein.

According to a another embodiment, the therapeutic regimen of the present invention comprises administering to a patient for at least about 4 weeks, more preferably either at least about 12 weeks or at least about 24 weeks:

(i) a therapeutically effective amount of Compound (1) or a pharmaceutically acceptable salt thereof once a day;
(ii) a therapeutically effective amount of interferon alpha once a week; and
(iii) a therapeutically effective amount of ribavirin twice a day.

An additional embodiment is directed to a packaged pharmaceutical composition comprising a packaging containing one or more doses of Compound (1) or a pharmaceutically acceptable salt thereof, an interferon alpha and ribavirin, together with written instructions directing the co-administration of Compound (1), an interferon alpha and ribavirin for the treatment of HCV infection in a patient that has been identified as having a non-CC genotype of SNP rs12979860 or a non-TT genotype of SNP rs8099917 located near the IL28B gene. The individual doses of Compound (1) or a pharmaceutically acceptable salt thereof, can be in the form of any of the standard pharmaceutical dosage forms, e.g. tablets, capsules, and packaged within any of the standard types of pharmaceutical packaging materials, e.g. bottles, blister-packs, etc., that may themselves be contained within an outer packaging material such as a paper/cardboard box. The written instructions will typically be provided either on the packaging material(s) itself or on a separate paper (a so-called “package insert”) that is provided together with the dosage forms within the outer packaging material. All such packaging embodiments and variations thereof are embraced by the present invention.

### Methods for Determining HCV Subtype and Subgenotypes

Specific methods that have been used for HCV RNA quantification, HCV subtyping and to IL28B genotyping are as detailed below. To the extent that other methods may be known and available in the art, and all are considered embraced within the present invention and can be used in connection therewith.

### HCV RNA Quantification

A plasma sample of about 6 ml is obtained from the patients and processed by using the Roche COBAS® TaqMan HCV/HPS assay. The assay has a linear range from 25 to 2000,000,000 IU/ml (2.0 E8 IU/ml) with a lower limit of quantification of 25 IU/ml and a lower limit of detection of 10 IU/ml.

### HCV Subtyping

The HCV subtype was determined by using the TRUGENE® HCV Genotyping Assay. The assay directly amplifies and sequences the virus allowing direct examination of the viral RNA by producing bi-directional sequences using two fluorescently-labeled DNA primers. The library includes viral isolates to allow determination of the 6 major hepatitis C genotypes and 41 sub-types.

### Genotyping of IL28B

Genotype analysis was performed on DNA extracted from blood samples of the patients by using TaqMan PCR based test assays established by Beckman Coulter Genomics (Bernried, Germany) for the analysis of genetic variants. The process flow of the genotype analysis consisted of the extraction of genomic DNA from blood samples, the application of established molecular genetic techniques to amplify the specific genetic target sites and the detection and analysis of emission data of the fluorescent TaqMan probes employed in the amplification processes. Three kinds of controls were used for each product: a) one water control included prior to DNA isolation, b) one water control included after DNA isolation and c) one heterozygous and/or one homozygous (wild-type or variant) genotyping control.

The process flow for TaqMan based products for allelic discrimination applied in this

[chemical structure]

study is given in the figure above. The final genotype results for all samples and all products of each processing batch were combined using the Beckman Coulter Genomics software SNPsuite. The results include information regarding the genotype of each subject.

### EXAMPLES

### I. Methods for Preparing Compound (1)

Methods for preparing amorphous Compound (1) can be found in U.S. Pat. Nos. 6,323,180, 7,514,557 and 7,585,845, which are herein incorporated by reference. Methods for preparing additional forms of Compound (1), in particular the crystalline sodium salt form, can be found in U.S. Pat. No. 8,232,293.

### II. Formulations of Compound (1)

One example of a pharmaceutical formulation of Compound (1) include an oral solution formulation as disclosed in WO 2010/059667. Additional examples include capsules containing a lipid-based liquid formulation, as disclosed in WO 2011/005646.

### III. Clinical Results

For the clinical trials described below, the Compound (1) drug product was administered as a softgel capsule filled with a lipid-based formulation containing Compound (1) sodium salt. All references to “Compound (1)” in the below clinical study is the sodium salt form.

### Example 6

### Clinical Study with Treatment-Naïve Patients

Treatment with the HCV protease inhibitor Compound (1) results in high and consistent SVR rates—results from SILEN-C1 in treatment naïve patients across different baseline factors

Background and Aims:

Compound (1) is a highly potent and specific HCV NS3/4A protease inhibitor given once daily (QD) with strong antiviral activity in chronic HCV genotype-1 (GT1) infection. This study presents a sub-group analyses of difficult-to-treat patients.

Methods:

In this double-blind, randomized phase II trial, 429 HCV GT1 treatment-naïve patients were randomized 1:1:2:2 to placebo or Compound (1), 120 mg with 3 days lead-in (LI) of pegylated interferon-alpha and ribavirin (PR) (120 mg QD/LI), 240 mg QD/LI or 240 mg QD without LI (240 mg QD). In each arm, Compound (1) was given for 24 weeks together with PR for 24 or 48 weeks. Viral load (VL) was measured by Roche TaqMan (lower limit of quantification 25 IU/mL), Subtype was assessed by NS3/4A sequencing, and IL-28B genotype (from the SNP rs12979860) was collected retrospectively on approximately 50% of patients. Based on slightly reduced response rates in both LI groups of Compound (1), the 240 mg QD dose has been selected for phase III evaluation (along with 120 mg QD). Comparison of this dose to PR across important patient characteristics are presented here.

### Main Inclusion Criteria

Age 18 to 65 years
GT-1 chronic hepatitis C infection
Naïve to interferon and/or RBV
HCV RNA≧100,000 IU/mL
Liver biopsy<2 years without cirrhosis

### Sub-Analyses Across Baseline Factors

HCV RNA assessed by Roche COBAS TaqMan® (lower limit of quantification: 25 IU/mL)
Subtype assessed by NS3/4A sequencing
IL28B genotype collected retrospectively on 50% of patients

Results:

Overall, 83.8% of patients treated with Compound (1) 240 mg QD achieved SVR, compared to 56.3% of patients on PR alone (p<0.0001). The SVR rates were consistently high across a wide range of difficult-to-cure subgroups (see Table). Notably, the SVR rate for subtype 1a patients was 82.4% (compared to 46.9% on PR, p=0.0013). All 22 patients (100%) with IL-28B genotype CC of SNP rs12979860 who received 24 weeks Compound (1) 240 mg without LI achieved SVR, compared with 81.8% for PR alone (p=0.1042). The SVR in non-CC (of SNP rs12979860) patients was also significantly higher (70.8%) compared to PR alone (41.4%, p=0.0162). The percentage of patients achieving eRVR and thus eligible for 24 weeks overall treatment duration was 87.3% with consistently high rates across all subgroups including non-CC patients.

| | | | | Compound (1) | | | |
|---|---|---|---|---|---|---|---|
| | | | | 240 mg QD | | | |
| | | Placebo | | (no Lead-in) | | | |
| | | | SVR | | eRVR\hat{ } | SVR | |
| Subgroup | | N | (%) | N | (%) | (%) | P-value* |
| | Overall | 71 | 56.3 | 142 | 87.3 | 83.8 | <0.0001 |
| Genotype | 1a | 32 | 46.9 | 51 | 78.4 | 82.4 | 0.0013 |
| | 1b | 38 | 65.8 | 91 | 92.3 | 84.6 | 0.0307 |
| IL-28B | CC | 11 | 81.8 | 22 | 95.5 | 100.0 | 0.1042 |
| | Non-CC | 29 | 41.4 | 48 | 85.4 | 70.8 | 0.0162 |
| | Missing | 31 | 61.3 | 72 | 86.1 | 87.5 | 0.0062 |
| Age | 18-40 | 21 | 66.7 | 38 | 76.3 | 86.8 | 0.0930 |
| | 40-55 | 39 | 56.4 | 72 | 90.3 | 81.9 | 0.0066 |
| | 55-66 | 11 | 36.4 | 32 | 93.8 | 84.4 | 0.0047 |
| BL ALT | Normal | 11 | 45.5 | 24 | 95.8 | 87.5 | 0.0146 |
| | >ULN | 60 | 58.3 | 118 | 85.6 | 83.1 | 0.0005 |
| BL GGT | Normal | 43 | 67.4 | 85 | 91.8 | 90.6 | 0.0022 |
| | >ULN | 28 | 39.3 | 57 | 80.7 | 73.7 | 0.0038 |
| BMI | <30 | 58 | 55.2 | 115 | 92.2 | 85.2 | <0.0001 |
| | ≧30 | 13 | 61.5 | 26 | 69.2 | 80.8 | 0.2535 |
| BL VL | <800,000 | 9 | 66.7 | 21 | 90.5 | 90.5 | 0.1432 |
| | ≧800,000 | 60 | 56.7 | 118 | 86.4 | 82.2 | 0.0005 |

\hat{ }eRVR = HCV RNA <25 IU/mL at Week 4 and undetectable at Weeks 8-20
ALT = alanine transaminase;
GGT, gamma-glutamyl transpeptidase;
ULN, upper limit of norm;
BL VL = baseline HCV RNA in IU/mL
*Fisher's exact test for equality of SVR rates

Of specific note is that the majority of patients with difficult to treat HCV subtypes, such as patients with the viral GT1a or the IL-28B non-CC gene variant (polymorphism), achieved SVR:
Specifically, among patients with GT1a HCV (n=51), a virus type that is more likely to resist treatment than GT1b, 82% achieved SVR, while for GT1b HCV patients (n=91), 85% achieved SVR
In addition, SVR was 71% for patients with the non-CC polymorphism of SNP rs12979860 located near the IL-28B gene (n=48), while patients with the CC polymorphism (n=22) achieved 100% SVR and those where IL-28B genotyping was missing (n=72) achieved 88% SVR. Patients exhibiting the non-CC polymorphism are less likely to achieve SVR with PegIFN/RBV treatment.

Conclusions:

Compound (1) 240 mg once daily in treatment naïve patients achieved consistently strong and significant eRVR and SVR rates, even in difficult-to-cure patients such as GT1a and IL28B non-CC. GT1 HCV is the most challenging genotype of HCV to treat (See, e.g., Ghany, Marc et al. “An Update on Treatment of Genotype 1 Chronic Hepatitis C Virus Infection: 2011 Practice Guideline by the American Association for the Study of Liver Diseases”, Hepatology, 54(4): 1433-44 (2011)) and those carrying a non-CC genotype of SNP rs12979860 or a non-TT genotype of SNP rs8099917 located near the IL-28B gene are less likely to achieve SVR than those with the CC or TT polymorphisms, with some studies showing an almost seven fold difference in treatment response. (See G. Cairns, “Gene variant that helps hepatitis C treatment may hinder HIV treatment”, 2011, at: http://www.bhiva.org/News.aspx?NewsID=a7503829-94b9-4d2f-bd91-1d2fbaad6e8d). Given this state of the art, the results obtained herein for the difficult to treat patient populations, including patients infected with HCV GT 1a and patients with a non-CC genotype of SNP rs12979860 located near the IL-28B gene reflect excellent results for this therapy. Based on the significantly reduced treatment effectiveness in such difficult-to-treat patient populations as seen with the combination of PegIFN/RBV and the NS3/4 HCV protease inhibitor telaprevir (Akuta et al. 2010), it is surprising that the expected IL28B-related difference in SVR rates observed in patients treated with PegIFN/RBV and telaprevir widely disappeared or were significantly reduced with the combination therapy of the present invention and that patients with the unfavorable genotypes non-CC or non-TT or with the unfavorable HCV genotype 1a infections achieved consistently high cure rates in terms of SVR.

The 240 mg once daily treatment is currently being tested along with 120 mg QD in phase III trials.

### Example 7

### Clinical Study in Japanese Patients

Title:

100% SVR in Japanese Genotype 1 Patients with IL28B TT Allele Treated with 4 Weeks Faldaprevir and 48 Weeks PegIFN/ribavirin

Background/Aims:

Phase II study results with Compound (1) (faldaprevir) and PegIFN/ribavirin (RBV) for Japanese chronic hepatitis C(CHC) patients was presented at APASL 2012. The aim of this analysis was to evaluate efficacy according to IL28B genotype in the Phase II cohort.

Methods:

The Phase II study was a randomised, double-blind, placebo-controlled trial investigating Compound (1) (faldaprevir) 120 mg and 240 mg once daily (QD) in treatment-naïve patients and 240 mg QD in an open-label group of treatment-experienced patients. In all groups, faldaprevir (or placebo) plus PegIFN/RBV was administered for 4 weeks, followed by PegIFN/RBV for 44 weeks. Among the 18 patients enrolled in the Phase II trial and administered faldaprevir, IL28B (rs8099917: TT, TG, or GG) genotyping was conducted on 15 patients (mean age 54.1±11.3 yr; 9 males, 6 females).

Results:

The IL28B major allele (TT) was found in 8 patients and the hetero/minor allele (TG/GG) was found in 7 patients. Sustained virological response (SVR) rates for TT and TG/GG were 100% (8/8) and 42% (3/7), respectively (p=0.026). Four patients whose IL28B was TG/GG could be assessed for HCV Core 70 amino acid substitution: all of them were R70Q, and the SVR was 25% (1/4).

### CONCLUSIONS

Treatment with 4 weeks faldaprevir plus PegIFN/RBV followed by 44 weeks PegIFN/RBV produced a high SVR rate in Japanese genotype 1 CHC patients with the IL28B major allele.

1. A method of treating hepatitis C viral (HCV) infection or alleviating one or more symptoms thereof in a patient comprising the step of administering to the patient a therapeutic combination comprising:
(a) a compound of the following formula (1) or a pharmaceutically acceptable salt thereof: [chemical structure]
wherein B is [chemical structure]
L¹ is MeO—; L¹ is Br; and R² is [chemical structure]
(b) interferon alpha; and
(c) ribavirin;
and wherein the patient has a non-CC genotype of SNP rs12979860 or a non-TT genotype of SNP rs 8099917 located near the IL-28B gene.

2. The method according to claim 1, wherein the patient has HCV subtype 1.

3. The method according to claim 1, wherein the patient has HCV subtype 1a.

4. The method according to claim 1, wherein the patient has a C/T genotype of SNP rs12979860 located near the IL-28B gene.

5. The method according to claim 1, wherein the patient has a T/T genotype of SNP rs12979860 located near the IL-28B gene.

6. The method according to claim 1, wherein the patient has a G/T genotype of SNP rs 8099917 located near the IL-28B gene

7. The method according to claim 1, wherein the patient has a G/G genotype of SNP rs 8099917 located near the IL-28B gene

8. The method according to claim 1, wherein said patient is a treatment-naive patient.

9. The method according to claim 1, wherein said patient is a treatment experienced patient.

10. The method according to claim 1, wherein the HCV-RNA levels of said patient are reduced to a less than detectable level as a result of the treatment.

11. The method according to claim 1, wherein said therapeutic combination is administered for at least 4 weeks.

12. The method according to claim 1, wherein said therapeutic combination is administered for at least 12 weeks.

13. The method according to claim 1, wherein said therapeutic combination is administered for at least 24 weeks.

14. The method according to claim 1, wherein compound (1) or a pharmaceutically acceptable salt thereof is administered at a maintenance dosage of at least 40 mg per day.

15. The method according to claim 1, wherein compound (1) or a pharmaceutically acceptable salt thereof is administered at a maintenance dosage between about 40 mg per day and about 480 mg per day.

16. The method according to claim 1, wherein compound (1) or a pharmaceutically acceptable salt thereof is administered at a maintenance dosage between about 120 mg per day and about 240 mg per day.

17. The method according to claim 1, wherein compound (1) is administered in the form of its sodium salt.

18. The method according to claim 1, wherein said ribavirin is administered at a dosage between about 200 mg/day and about 1800 mg/day.

19. The method according to claim 1, wherein said ribavirin is administered at a dosage between about 800 mg/day and about 1200 mg/day.

20. The method according to claim 1, wherein said interferon alpha is a pegylated interferon alfa.

21. The method according to claim 20, wherein said pegylated interferon alfa is pegylated interferon alfa-2a or pegylated interferon alfa-2b.

22. The method according to claim 20, wherein the pegylated interferon alfa is pegylated interferon alfa-2b administered at a dosage of about 0.5 μg/kg/week to about 2 μg/kg/week.

23. The method according to claim 20, wherein the pegylated interferon alfa is pegylated interferon alfa-2b administered at a dosage of about 1 μg/kg/week to about 2 μg/kg/week.

24. The method according to claim 20, wherein the pegylated interferon alfa is pegylated interferon alfa-2b administered at a dosage of about 1.5 μg/kg/week.

25. The method according to claim 20, wherein the pegylated interferon alfa is pegylated interferon alfa-2a administered at a dosage of about 90 to 200 μg/week.

26. The method according to claim 20, wherein the pegylated interferon alfa is pegylated interferon alfa-2a administered at a dosage of about 180 μg/week.

27. The method according to claim 1, wherein the HCV infection is subtype 1, the patient is a treatment experienced patient, the compound (1) or a pharmaceutically acceptable salt thereof is administered at a maintenance dosage between about 120 mg per day and about 240 mg per day and wherein said interferon alfa is pegylated interferon alfa -2a or pegylated interferon alfa-2b.

28. The method according to claim 27, wherein compound (1) is administered in the form of its sodium salt.

29. The method according to claim 1, wherein the patient has first been identified as having a non-CC genotype of SNP rs12979860 or a non-TT genotype of SNP rs 8099917 located near the IL-28B gene prior to the administration step.

30. A packaged pharmaceutical composition comprising a packaging containing:
(a) one or more doses of the following formula (1) or a pharmaceutically acceptable salt thereof: [chemical structure]
wherein B is [chemical structure]
L⁰ is MeO—; L¹ is Br; and R² is [chemical structure]
and (b) written instructions directing the co-administration of Compound (1), or a pharmaceutically acceptable salt thereof, interferon alpha, and ribavirin for the treatment of HCV infection in a patient that has a non-CC genotype of SNP rs12979860 or a non-TT genotype of SNP rs 8099917 located near the IL-28B gene.
